(19) United States
(12) Patent Application Publication
(10) Pub. No.: US 2024/0353298 A1
(43) Pub. Date: Oct. 24, 2024

(54) SAMPLE COLLECTION APPARATUS

(71) Applicant: Kenota Inc., Kitchener (CA)

(72) Inventors: Christopher J. Harder, Osborne Corner (CA); Rob J. Rennie, Stratford (CA); Adam D. Pyke, Hamilton (CA); Calvin James Brereton, Kitchener (CA)

(21) Appl. No.: 18/303,678

(22) PCT Filed: Oct. 25, 2021
(86) PCT No.: PCT/CA2021/051498

§ 371 Date: Apr. 20, 2023

(51) Int. Cl. G01N 1/40

(52) U.S. Cl. CPC G01N 1/4077; G01N 2001/4088

(57) ABSTRACT

A sample collection apparatus for fluid handling of small amounts of sample fluid and loading of a syringe. The present sample collection apparatus can be utilized as a precise dispense device within an accompanying instrument, such as an input to an analytical instrument, point-of-care device, or automated analyzer. The present apparatus can also be used for preparing whole blood samples and for separating plasma from the cellular components of whole blood without centrifugation, as well as for providing filtered samples of biological materials.

### CROSS-REFERENCE TO RELATED APPLICATIONS

This application claims priority to United States provisional patent application U.S. 63/105,415 filed 26 Oct. 2020, and is a PCT National Entry of PCT/CA2021/051498 filed on 25 Oct. 2021, both of which are hereby incorporated by reference herein in their entirety.

### FIELD OF THE INVENTION

The present invention pertains to a fluid handling and sample collection apparatus for directing small amounts of fluid into the tip of a microsyringe. The present invention also pertains to a device for controlled loading of a microsyringe with a small amount of sample fluid for use in automated analysis of the fluid.

### BACKGROUND

Biological fluids for laboratory testing are generally collected in the minimal volume necessary to carry out the test required. For blood tests in particular, it is desirable to be able to provide diagnostic results using small amounts of blood such to limit the exposure and technical requirement for phlebotomy needed for collecting larger amounts of blood. For quantitative tests in particular, collection of a sufficient amount as well as standardizing the measured aliquot of blood volume for the quantitative test improves the accuracy of the test. In cases where the quantitative testing is automated, accurate delivery of a known volume of blood ensures less error in the quantitative results and higher overall accuracy.

In the preparation of small volumes of fluid for testing in an automated device such as a point-of-care device, the injection of a reliably known volume of biological fluid contributes to the overall quality and reproducibility of the quantitative test. To ensure that the small volume of biological fluid to be tested is standardized, the fluid should be free of bubbles and air pockets in the injection device, otherwise the results of the test will be quantitatively lower than otherwise would have observed with the full amount of fluid. To ensure standardized fluid injection at low volumes of fluid, such as that in the 0.1-50 microlitre range, the automated injection device should be loaded to prevent disturbance of fluid flow to prevent volume injection inaccuracies. Small scale whole blood handling can also be advantageous for point-of-care testing devices used to provide an efficient and quick blood component analysis at or near the patient to make immediate and expedient decisions about patient care. Point-of-care testing is generally performed by untrained or limitedly trained laboratory personnel, and simplicity of sample preparation can limit errors and enable improved results. An easy and reproducible fluid preparation process facilitates rapid blood analysis and improves operating conditions for point-of-care devices which generally require only small amounts of fluid volume.

Various devices are available for obtaining very small amounts of plasma at the Point of Care (PoC) by separating plasma from whole blood by means of filtering. In one example, United States patent U.S. Pat. No. 9,283,313 to Huemer describes a multi-part device for separating plasma from whole blood having a filter unit for extracting plasma and a pumping unit for creating a partial vacuum in the filter unit.

Other devices are available for small volume fluid handling, such as United States patent U.S. Pat. No. 9,833,382 to Lin et al. which describes a needle filter apparatus needle seal stopper and needle stop to create a substantially airtight chamber to facilitate fluid aspiration through an enclosed filter and into the syringe. In this case, the syringe is used for aspiration of fluid such as medicament or drug-laden solutions up into a syringe in an airtight manner for filtering solution to effectively eliminate fluid contaminants from being drawn into a syringe.

There remains a need for a device for preparing small volume fluid samples which is simple, safe, and economical to handle and provides a fluid sample suitable for small volume injection into an automated analyzer.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide a sample collection apparatus which can process small amounts of sample fluid to fill a syringe. Another object of the present invention is to provide a sample collection apparatus and method for preparing a syringe for small volume fluid injection in an automated analyzer.

In an aspect there is provided a sample collection apparatus comprising: a filling assembly comprising: an inlet port for receiving a sample fluid; an outlet port fluidly connected to the inlet port by a fluid channel; a receiving syringe having a first end with an aperture for engaging with the filling assembly and a second end comprising an injection needle; a fluid conducting element extending from the outlet port of the filling assembly toward the second end of the receiving syringe adjacent the injection needle; and a pressure release structure in the receiving syringe to release pressure in the receiving syringe as sample fluid flows via the fluid conducting element. In another aspect there is provided a syringe loading apparatus comprising: a housing comprising: an inlet port comprising a fluid channel for receiving biological fluid; an outlet port fluidly connected to the inlet port; a fluid channel structure fluidly connecting the inlet port and the outlet port; and a syringe engagement feature; a receiving syringe having a first end with a flange for releasably attaching and aligning with the syringe engagement feature of the housing and a second end comprising a syringe needle; a microtube extending from the outlet port of the housing to the second end of the receiving syringe adjacent the syringe needle; and a pressure release structure in the receiving syringe.

In another aspect there is provided a syringe loading apparatus comprising: a housing comprising: an inlet port comprising a fluid channel for receiving biological fluid; an outlet port fluidly connected the inlet port; a fluid channel structure fluidly connecting the inlet port and the outlet port; and a flange locking feature; a receiving syringe having a first end with a flange for releasably attaching and aligning with the flange locking feature of the housing and a second end comprising a syringe needle; a microtube extending from the outlet port of the housing to the second end of the receiving syringe adjacent the syringe needle; and a pressure release structure in the receiving syringe.

In an embodiment, the inlet port is sized to fluidly connect with a diluent syringe for receiving diluent under pressure.

In another embodiment the apparatus further comprises a filter in the housing disposed between the inlet port and the outlet port.

In another embodiment, the receiving syringe volume is 1 mL or less.

In another embodiment, the receiving syringe diameter is 4 mm or less.

In another embodiment, the inlet port is tapered.

In another embodiment, the fluid channel structure in the housing is non-linear.

In another embodiment, the microtube is made from polymer, metal, or a combination thereof.

In another embodiment, the fluid channel structure in the housing further comprises a flow restrictor.

In another embodiment, the fluid conducting element comprises a hollow shaft.

In another embodiment, the fluid conducting element comprises a channel guide which, together with a sidewall of the receiving syringe, forms a fluid channel.

In another embodiment, the microtube bore size is between about 0.05 and 3.0 mm.

In another embodiment, the filling assembly engages with the receiving syringe via a friction fit feature, snap-fit feature, a flange-locking feature, or a combination thereof.

In another aspect there is provided a kit comprising: a sample collection apparatus comprising: a housing comprising: an inlet port comprising a fluid channel for receiving biological fluid; an outlet port fluidly connected the inlet port; a fluid channel structure fluidly connecting the inlet port and the outlet port; and a syringe engagement feature; a receiving syringe having a first end with a flange for releasably attaching and aligning with the syringe engagement feature of the housing and a second end comprising a syringe needle; a microtube extending from the outlet port of the housing to the second end of the receiving syringe adjacent the syringe needle; and a pressure release structure in the receiving syringe; and diluent syringe comprising diluent.

In another aspect there is provided a sample collection kit comprising: a filling assembly comprising: an inlet port for receiving a sample fluid; an outlet port fluidly connected to the inlet port by a fluid channel; a receiving syringe having a first end with an aperture for engaging with the filling assembly and a second end comprising an injection needle, and an inside wall; a fluid conducting element extending from the outlet port of the filling assembly toward the second end of the receiving syringe adjacent the injection needle; and a pressure release structure in the receiving syringe to release pressure in the receiving syringe as sample fluid flows via the fluid conducting element; and a microplunger having a plunger tip for creating a fluid-tight seal with the inside wall of the receiving syringe.

In an embodiment, the kit further comprises a fluid sample transfer pipette having a tip configured to fit inside the inlet port of the filter housing.

In another embodiment, the kit further comprises a lancet.

In another aspect there is provided a method of separating plasma from whole blood comprising: applying a blood sample to a filter; applying a diluent to the filter under pressure; directing diluted plasma downstream the filter through a microtube; and collecting the diluted plasma in a receiving syringe adjacent a syringe needle in the receiving syringe.

In an embodiment, the method further comprises ejecting the diluted plasma through the syringe needle into a diagnostic device.

### BRIEF DESCRIPTION OF THE FIGURES

For a better understanding of the present invention, as well as other aspects and further features thereof, reference is made to the following description which is to be used in conjunction with the accompanying drawings, where:

FIG. 1 is an example of a sample collection apparatus;

FIG. 2 is a side view of a sample collection apparatus with engaged pipette;

FIG. 3 is a side cross-sectional view of a sample collection apparatus with engaged pipette;

FIG. 4A is a front view of a filling assembly with engaged fluid conducting element;

FIG. 4B is a closeup view of a fluid conducting element and receiving syringe;

FIG. 5A is a side cross-sectional view of an embodiment of a two-piece filling assembly;

FIG. 5B is an isometric view of the bottom piece of a filling assembly shown in FIG. 5A;

FIG. 6A is a side view of a two-piece filling assembly with engaged pipette;

FIG. 6B is a side perspective view of a filling assembly top;

FIG. 7 is a side cross-sectional view of a filling assembly with engaged transfer pipette;

FIG. 8 is an image of a whole blood and filtered blood sample;

FIG. 9A is a side perspective view of a sample collection apparatus with an engaged diluent syringe;

FIG. 9B is a side perspective transparent view of a sample collection apparatus with an engaged diluent syringe;

FIG. 10A is a side perspective exploded view of a receiving syringe with a pressure release structure;

FIG. 10B is a side perspective transparent view of a receiving syringe with a pressure release structure inside the syringe;

FIG. 11A is a side view of a sample collection apparatus with syringe flange engagement features;

FIG. 11B is a side cross-sectional view of a sample collection apparatus with syringe flange engagement features;

FIG. 12A is a cross-sectional view of a filter assist apparatus;

FIG. 12B is a close-up cross-sectional view of a filter assist apparatus;

FIG. 13 is a side cross-sectional view of an alternative embodiment of a sample collection apparatus;

FIG. 14 is a side cross-sectional view of an alternative embodiment of the sample collection apparatus with a microplunger and sample transfer pipette;

FIG. 15 is a top isometric view of a one-piece fluid conducting element and filling assembly;

FIG. 16 is a bottom isometric view of a one-piece channel guide and filling assembly;

FIG. 17 is a cross-sectional view of a channel guide serving as a fluid conducting element in a receiving syringe;

FIG. 18A is a side cross-sectional view of a receiving syringe with engaged microplunger;

FIG. 18B is a side cross-sectional view of a receiving syringe with engaged microplunger and injection rod;

FIG. 18C is a side cross-sectional view of a receiving syringe with engaged microplunger and injection rod with the microplunger head below the end of the pressure release structure;

FIG. 18D is a side cross-sectional view of a receiving syringe with engaged microplunger and injection rod in a fully deployed state;

FIG. 19 is a top isometric view of a receiving syringe with attachment clip for holding a microplunger; and

FIG. 20 is a graph displaying measured IgE results from Example 1.

### DETAILED DESCRIPTION OF THE INVENTION

Unless defined otherwise, all technical and scientific terms used herein have the same meaning as commonly understood by one of ordinary skill in the art to which this invention belongs.

As used in the specification and claims, the singular forms “a”, “an” and “the” include plural references unless the context clearly dictates otherwise.

As used herein, the terms “comprising.” “having.” “including” and “containing.” and grammatical variations thereof, are inclusive or open-ended and do not exclude additional, unrecited elements and/or method steps, and that that the list following is non-exhaustive and may or may not include any other additional suitable items, for example one or more further feature(s), component(s) and/or element(s) as appropriate. A composition, device, article, system, use, process, or method described herein as comprising certain elements and/or steps may also, in certain embodiments consist essentially of those elements and/or steps, and in other embodiments consist of those elements and/or steps and additional elements and/or steps, whether or not these embodiments are specifically referred to.

As used herein, the term “about” refers to an approximately +/−10% variation from a given value. It is to be understood that such a variation is always included in any given value provided herein, whether or not it is specifically referred to. The recitation of ranges herein is intended to convey both the ranges and individual values falling within the ranges, to the same place value as the numerals used to denote the range, unless otherwise indicated herein.

The use of any examples or exemplary language, e.g. “such as”, “exemplary embodiment”, “illustrative embodiment” and “for example” is intended to illustrate or denote aspects, embodiments, variations, elements or features relating to the invention and not intended to limit the scope of the invention.

As used herein, the terms “connect” and “connected” refer to any direct or indirect physical association between elements or features of the present disclosure. Accordingly, these terms may be understood to denote elements or features that are partly or completely contained within one another, attached, coupled, disposed on, joined together, in communication with, operatively associated with, etc., even if there are other elements or features intervening between the elements or features described as being connected.

The term “plasma” as used herein refers to the liquid component of blood that normally holds the blood cells in whole blood in suspension. When separated from blood cells, plasma comprises dissolved and suspended substances including gasses, biochemicals and inorganic salts and minerals. In contrast, the term “serum” refers to plasma from which the clotting proteins have been removed.

As used herein, the “sample fluid” refers to any fluid desired for downstream use, for example in an analytical method. Non-limiting example of fluids which may be processed using the present device include biological fluids such as human or animal bodily fluids like blood, plasma, serum, lymph, urine, saliva, semen, amniotic fluid, gastric fluid, phlegm, sputum, mucus, tears, stool, etc. Other types of samples that can be processed can be derived from human or animal tissue samples where the tissue sample has been processed into a liquid, solution, or suspension to reveal particular tissue components for examination. Other non-limiting examples of samples that can be processed as sample fluid are environmental fluid samples, food industry fluid samples, and agricultural fluid samples.

Herein is described a sample collection apparatus which can be used for fluid handling of small amounts of sample fluid and loading of a syringe. Sample injected into the present sample collection device through a transfer pipette is directed adjacent to a small diameter, low volume injection needle in a substantially air-free manner such that the process substantially eliminates introduction of bubbles and air pockets which can later interfere with quantitative sample injection. This is particularly important in the standardized dispense of accurate and repeatable microlitre-scale sample volumes. The present sample collection apparatus can thereby be utilized as a precise dispense device within an accompanying instrument, such as an input to an analytical instrument, point-of-care device, or automated analyzer. The input to the accompanying instrument can be either diluted or non-diluted based on the required or desired input of the instrument and analytical process desired for sample preparation. It is also understood that the present sample collection apparatus can be used to filter particulate and cells from other biological fluids and samples where filtration and dilution are desired.

The present invention also provides a device and method for separating cells from the plasma fraction of blood without centrifugation by separating and isolating plasma from the cellular components of whole blood. Small amounts of blood can be separated, providing diluted or non-diluted plasma for laboratory testing. Diluted plasma obtained using the present device can be utilized for small scale point-of-care laboratory testing for the detection of the presence of chemicals, nutrients, proteins, biological molecules, and particulate less than cellular size which is normally dissolved or suspended in plasma. The diluted plasma collected is substantially cell-free, and cells from the collected whole blood are retained by the filter. For small amounts of blood, the present device can be used to achieve separation of the blood components in a short time and with an apparatus of minimum complexity that can be used safely by a relatively unskilled person, while obtaining diluted plasma-carried molecules suitable for immediate use in a point-of-care diagnostic.

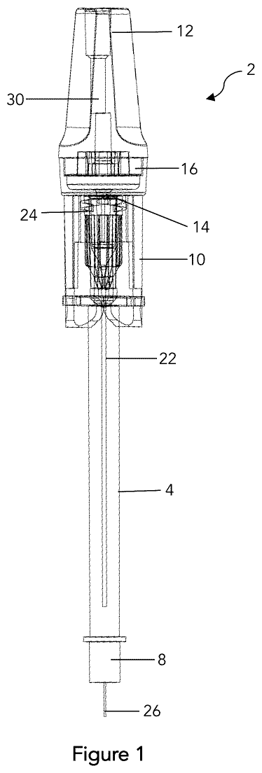

FIG. 1 is a sample collection apparatus 2 comprising a filling assembly 10 for receiving sample fluid and a fluid conducting element 22 for directing the biological fluid from the filling assembly 10 and into the bottom of and receiving syringe 4 for collecting the biological fluid. The filling assembly 10 can be comprised of one or more pieces that are releasably or permanently engaged together, and comprises an inlet port 12 for receiving the sample fluid and an outlet port 14 for directing the fluid into the receiving syringe 4. The sample fluid is optionally diluted during syringe filling using a diluent which can be applied in the inlet port 12 after the sample fluid has been injected into the filling assembly 10.

Receiving syringe 4 has a syringe flanges 6 which can optionally engage with the filling assembly 10. The attachment between receiving syringe 4 and filling assembly 10 can be a permanent or releasable connection, and the filling assembly 10 can optionally comprise one or more syringe syringe engagement feature s for secure and releasable attachment and engagement of the receiving syringe 4 with the filling assembly 10. In other embodiments, the filling assembly 10 assembly can be releasably attached to the filling syringe 4 via friction fit or other engagement feature.

The filling assembly can optionally be fitted with a filter element disposed between the inlet port 12 and outlet port 14 of the filling assembly 10, or between the inlet port 12 and the fluid conducting element 22, to filter the sample fluid prior to filling the receiving syringe 4 through the fluid conducting element 22. The dimensions of the filling assembly can vary, and can accommodate an optional filter disc diameter in the range of from, for example, 1 mm-40 mm. The inlet port 12 in the filling assembly 10 is configured for receiving and/or engaging with a micropipette, pipette, or other syringe for delivering biological fluid to the filling assembly. The inlet port 12 can further comprise a releasable locking mechanism to engage a pipette or syringe, such as, for example, a friction fit mechanism, snap fit mechanism, screw-type lock, or luer-lok®.

Different configurations of the fluid channel 30 between the inlet port 12 and outlet port 14 of the filling assembly 10 can be designed to adjust the fluid path-length and fluid path direction through the filling assembly 10 to achieve a substantially air-free filling. The fluid channel 30 can be, for example, a straight channel, non-linear channel, or can have any suitable configuration for directing biological fluid toward the microtube 22. In one configuration a non-straight fluid channel directs fluid to prevent sample fluid and/or diluent from being applied to or sprayed into the filling assembly 10 with a high pressure stream which can embed coagulated or large particulate into the filter and impede flow and damage any filter in the filling assembly 10. A flow restrictor or channel having a flow restricting feature can be any device and/or structure that provides a non-straight fluid path in the fluid channel, such as a bend or channel narrowing, for example. The flow restrictor can enable application of the sample fluid to a sidewall of the fluid channel instead of the main body of the filling assembly. By controlling the flow rate and flow direction, the integrity of the sample fluid can also be controlled and the introduction of air during the filling can be limited. In addition, controlling the rate, direction, and pressure of flow of the sample fluid prevents damage to any filter membrane in the filling assembly as filtration rate can affect the quality of the filtrate. Narrowing the liquid path or fluid channel also forces limited and controlled mixing in the case of diluent use, resulting in more consistent fluid mixing conditions and more standardized mixing. In the use of the present device for separation of plasma from blood, narrowing the fluid path or providing a flow restricted fluid path can limit mixing of the blood with any buffer or diluent loaded after and provide a more concentrated plasma output.

Shown in FIG. 1 is an axially offset two-part fluid channel 30 through the filling assembly 10, which is one optional configuration of the fluid channel. The configuration of the liquid path also forces sequential loading of the biological fluid and diluent so as to not excessively dilute the sample when filtering, which can provide a concentrated sample filtrate for downstream analytical use. Other fluid channel configurations are possible, including but not limited to straight, arced, corkscrew, length, or other fluid channel configurations. The fluid channel can further vary by diameter, or can be of variable diameter in an individual channel.

In the embodiment shown in FIG. 1, fluid conducting element 22 comprises a microtube extending from the filter outlet port 14 on the filling assembly 10, and is engageably attached to the filter outlet port 14 through microtube hub 24. The attachment of the microtube hub 24 to the filter outlet port 14 can be, for example, snap fit, friction fit, threaded, or other type of fit, and can be permanent or non-permanent. The microtube shown is a long tube shaft extending from a microtube hub 24 on the filling assembly to direct fluid from the filter outlet port 14 to the bottom of the syringe. The microtube can be made from polymer, metal, or other material capable of retaining a tubular shape including but not limited to polypropylene, polyethylene, acrylonitrile butadiene styrene (ABS), or other rigid polymers, and should be of a large enough diameter such that fluid pressure applied to the filter when diluent is applied to the filter under pressure, fluid passes through the filter and is directed through the microtube 22 to the syringe tip. In an example, the microtube can have an inner diameter of inner diameter 0.05-3.0 mm, with preferable inner diameter between about 0.2 mm and 2 mm.

The receiving syringe 4 receives fluid through the microtube into syringe tip 8. As shown, the microtube is attached to and in fluid communication with the outlet port 14 of the filling assembly 10 for receiving fluid or diluted fluid from the filling assembly 10. The receiving syringe 4 has a barrel length that is the same length or slightly longer than the end of the fluid conducting element 22 or microtube such that effluent fluid from the microtube is received in the syringe tip 8 adjacent the injection needle 26 to limit inclusion of air in the sample fluid collected near the injection needle 26. The receiving syringe 4 preferably ranges in volume from about 0.3 mL to 10 mL.

Syringe tip 8 receives optionally diluted sample fluid which has been directed through the fluid channel 30 in the filling assembly 10 and the microtube. An injection needle 26 on syringe tip 8 serves as an injection device for the micro-injection of collected fluid into an automation apparatus for analysis. The injection needle 26 can be beveled or unbeveled and is preferably made from plastic or metal with a diameter of between about 0.05 mm and 3 mm. The receiving syringe 4 through injection needle 26 can be fitted onto a point-of-care, automation apparatus, or laboratory device, to apply microlitre amounts of sample fluid to an assay device. An optional needle cap for the injection needle 26 can be provided to protect the injection needle 26 from damage during sample processing and also to protect the technician from accidental injury from the injection needle.

The filling assembly 10 preferably comprises at least one syringe engagement feature 18 for secure attachment to the syringe to provide a fluid connection from the fluid channel to the fluid conducting element 22. The one or more syringe engagement feature 18 on the filling assembly 10 can releasably or permanently secure the syringe flanges 6 to the filling assembly 10. Two syringe engagement features 18 are shown, which in this embodiment engage with the syringe flanges to create a tight friction fit between the filling assembly 10 and receiving syringe 4, however other configurations of syringe engagement features are conceivable including snap and friction fit engagement features.

An optional filter 16 can be placed inside the fluid channel 30 or adjacent the outlet port of the filling assembly 10 comprising a filter membrane for retaining particulate or cells desired for exclusion from the fluid which enters fluid conducting element 22. When the sample collection apparatus is being used for separating whole blood, the filter membrane in the filter housing receives the whole blood, and then the filter membrane is washed with diluent or buffer to extract the plasma and small molecules dissolved and suspended therein that are not retained by the filter. Other sample fluids which benefit from pre-filtering include environmental samples to filter out insoluble particulate. The filter membrane may be woven or non-woven and having pore sizes selected to separate undesirable particulate from the desired sample fluid for downstream assay or analysis. For example, blood cells can be separated from other blood components, such as cells from plasma, to collect the plasma only for analysis. The filtration means can also be selected to filter out a particular molecule size range so that only a particular size range of molecule is present in the filtrate. Blood cells are generally 3 μm or larger, so as long as cell lysis is minimized and cells are substantially stabilized the filter will substantially exclude cellular matter from the collected diluted plasma. As plasma also comprises lysed cell matter, isolating all cellular matter from the collected diluted plasma is not possible, however limiting the amount of cellular matter in the collected plasma can improve the quality of analytical testing, especially in the case of testing for presence and levels of plasma-carried antibodies. The filter should be made from a suitable membrane material with an appropriate pore size and membrane thickness to retain the desired particulate matter or cells. The filter 16 may also comprise multiple membrane types with the same or different thickness and the same or different fibre. Suitable membrane fibers can include naturally derived materials as well as synthetic materials including but not limited to polytetrafluoroethylene (PFTE), Polyvinylidene fluoride (PFDF), polycarbonate, nylon, polysulfone, cellulose, and nitrocellulose. Various filter structures can also include amorphous fibrous filters, crystalline filters, graphene, and other filter types.

The relatively small size of the present sample collection apparatus provides portability for the technician preparing a sample as well as minimizes waste produced in the sample preparation process. The sample collection apparatus is also preferably sized and shaped and dimensioned so as to be convenient to hold and manipulate by the technician. The apparatus will normally be a single-use disposable item, though it is also conceivable to have the sample collection apparatus fitted with parts that can be swapped out and/or sterilized and cleaned for multiple uses. Devices to secure the sample collection apparatus, such as a holding device to maintain the sample collection device upright while loading can also be envisaged and provided.

In use with preparation of a blood sample, a sample of blood, preferably between about 0.01 mL and 2.0 mL, is applied through the inlet port into the fluid channel 30 and received into the body of filling assembly 10. The blood is then pushed through the fluid channel 30 structure under pressure and/or diluent is applied to the inlet port 12 under pressure to push diluent through the filter, directing plasma through the microtube and into the bottom end of receiving syringe 4 and retaining cells on the filter. Receiving syringe 4 along with fluid conducting element 22 is then removed from the filter housing and a piston or microplunger is put into the syringe to eject collected plasma or diluted plasma through injection needle 26, preferably under precise control using an automated diagnostic device. Consistent delivery of a known small volume of fluid to the analytical device under precise ejection control provides improved accuracy and reproducibility to enable quantitative concentration measurements of components in the fluid.

The closed transfer of fluids in the sample collection apparatus provides substantially leak-proof sealing and pressure equalization during engagement of the device while shielding biological and other sample fluids from contamination and providing protection to technicians using the device. Transfer of blood or other biological fluids from a collector through the inlet port 12 can be accomplished with minimal manipulation and minimal exposure of the sample fluid to air or laboratory. The leak-proof sealing of the system substantially prevents inadvertent leakage of both air and liquid during use of the system. Containment of biological fluid once collected can be entirely contained within the sample collection apparatus, preventing contamination of sample, technician, and laboratory.

The system of the present disclosure can also permit pressure equalization in the syringe between an attached diluent syringe, sample pipette, or sample syringe when the sample collection apparatus is attached. In one embodiment, a pressure relief hole or aperture can be located in the syringe device to provide air release during pressurization. The location of the pressure equalization feature or pressure release structure can be changed based on the volume of the sample collection apparatus and on the volume of fluid being filtered. In this way, a fluid column can be pushed down by the plunger in the device to void the air under the column of liquid without pressure buildup in the device.

Agglomeration control substances can also be added to the syringe filling device 2 or sample fluid either prior to or during operation of the device to control agglomeration and/or coagulation of components in the sample fluid. In one example, agglomeration control can prevent clogging of the fluid conducting element 22, in this example a microtube, and injection needle 26 and provide consistency in fluid volume delivery to an analytical device. In another example in the use of blood, it has been found that controlled agglomeration of blood matter into particle sizes large enough not to be able to process through a filter during filtration but small enough so as not to clog the membrane or fluid channels can be useful to exclude cells and large molecules and obtain improved samples of plasma containing smaller and lower molecular weight molecules found in blood, such as proteins, sugars, mineral ions, hormones, suspended and dissolved biochemicals, IgE antibody molecules, and nutrients. In some uses, it is desirable that particulates larger than 3 μm, or between 3 μm-200 μm will preferably be retained by the filter in a form that it does not clog up the filter. Agglomeration control substances can be added or embedded into elements of the sample collection apparatus 2 itself, such as to the filling assembly 10 such as to the fluid flow channels, or to the optional filter, to control coagulation or agglomeration of the fluid during fluid handling. Various types and concentrations of anticoagulant can be used. Some agglomeration control substances that may be used include but are not limited to sodium citrate, ethylene diamine tetraacetic acid (EDTA), thrombin, coumarins, heparin and heparin-like compounds, and anti-glycophorin A (which is used to agglutinate red blood cells).

Although use of the present device is described as primarily being applied to biological fluid handling including whole and separated blood, it is understood that the presently described sample collection apparatus can be used on a wide variety of biological and non-biological sample fluids to separate large particulate or cells from dissolved and suspended smaller matter which can pass through the filter membrane. Some examples of fluids which may be processed using the present device include but are not limited to human or animal bodily fluids such as blood, plasma, serum, lymph, urine, saliva, semen, amniotic fluid, gastric fluid, phlegm, sputum, mucus, tears, stool, etc. Other types of samples that can be processed are derived from human or animal tissue samples where the tissue sample has been processed into a liquid, solution, or suspension to reveal particular tissue components for examination. Other non-limiting examples of samples that can be used are environmental samples, food industry samples, and agricultural samples.

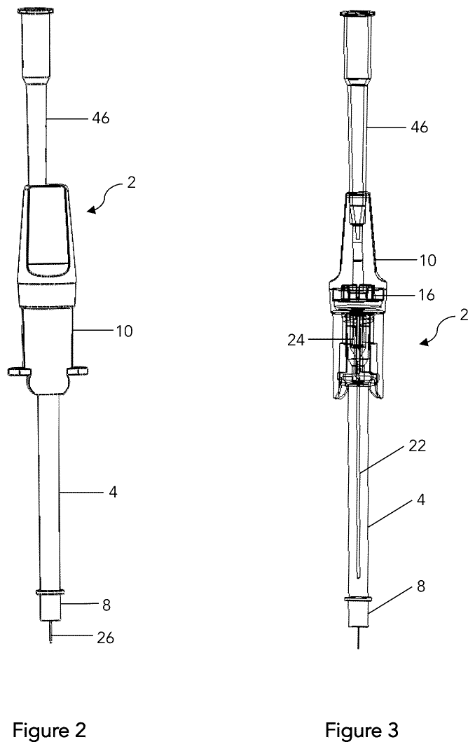

FIG. 2 is a side view of a sample collection apparatus 2 with engaged sample collection apparatus. During collection of small amounts of blood or other sample fluid, such as from a patient, a transfer pipette can be used to get hygienic and secure sample transfer between patient or sample source and processing, diagnostic, and test equipment. A transfer pipette 46 is a small pipette that can be used for collecting small volumes of fluids, and is generally less than about 1 mL. Transfer pipettes can collect fluid by capillary action or suction, where the suction can be applied by a vacuum, bulb, or syringe barrel, for example, and can dispense sample fluid using applied pressure or capillary action. A transfer pipette 46 can optionally also comprise a barrier or filter and/or an anti-agglomerant substance or coating to prevent clogging and fluid agglomeration. As many analytical assays can be used with only minute amounts and concentrations of biological or sample fluids, drops of blood can be collected from a finger prick and sufficient material can be obtained for analysis of, for example, antibodies in blood. The blood can then be applied from the transfer pipette to the filter inlet port inside the filling assembly 10 for processing. During application of the biological fluid to the filling assembly, the receiving syringe 4 is attached to the filling assembly 10 such that any diluent later put through the fluid channel in the filling assembly 10 and optional filter under pressure will force the diluted or undiluted sample into the syringe tip 8 adjacent the injection needle 26. Injection needle 26 is provided, optionally with a cap, to later engage with an analytical device and/or is used to meter out diluted plasma sample onto the analytical device.

FIG. 3 is a side cross-sectional transparent view of a sample collection apparatus 2 with engaged transfer pipette 46. Once sample fluid is collected by the transfer pipette or other collection device it is applied to the filling assembly 10 and optional filter 16 via a fluid channel in the filling assembly 10. In the embodiment shown, microtube hub 24 connects the filter outlet port in the filling assembly to the fluid conducting element 22, which in this embodiment is a microtube, directing fluid through the filling assembly 10 and optionally filtered by the filter to the syringe tip 8 for preparing for injection of sample fluid into an analytical device.

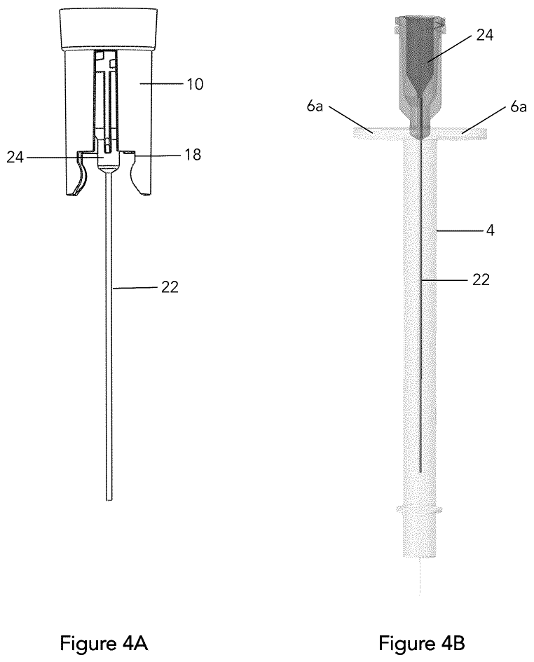

FIG. 4A is a front view of a filling assembly 10 with an engaged microtube which serves as a fluid conducting element 22 inside the receiving syringe. The microtube shaft is attached to the filling assembly 10 through microtube hub 24, which is attached to the outlet port of the filling assembly 10 such that it can withstand the fluid pressure applied to flush the fluid channel and optional filter while directing fluid to the opposite end of the syringe into the syringe tip for controlled fluid collection without air bubble introduction into the fluid. Filling assembly 10 is shown with syringe engagement feature 18 which engage with the syringe to provide a secure fluid collection. Syringe engagement features on the filling assembly also provide secure placement and attachment to a receiving syringe. An air channel is preferably created between filling assembly 10 and the receiving syringe such that pressure can be equalized in the sample collection apparatus during high pressure application of through the fluid channel structure and via fluid conducting element 22. Although in some cases a near complete seal may be desired, equalization of air pressure by providing an incomplete seal between the syringe and filling assembly can prevent pressure accumulation in the sample collection apparatus. Design of an incomplete seal to allow air exchange and eliminate pressure or air build-up inside the syringe can include one or more holes or apertures, optionally created or supported by a structure such as, for example, an aperture support structure or blocking structure such as a pressure release structure to maintain and allow air flow.

FIG. 4B is a closeup view of a microtube serving as a fluid conducting element 22 and receiving syringe 4 with the microtube shaft fitted inside the receiving syringe 4 to direct fluid to the bottom of the syringe. Syringe flanges 6a and 6b can engage with syringe engagement features on the filling assembly for correct and secure placement of the syringe relative to the microtube and filling assembly.

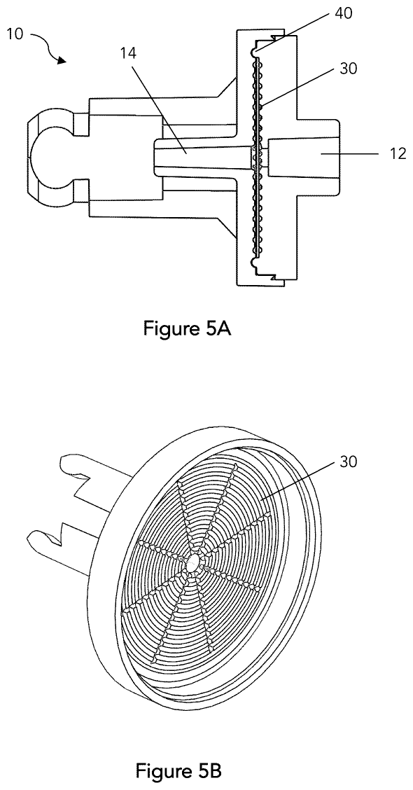

FIG. 5A is a side cross-sectional view of an embodiment of a two-piece filling assembly 10 which fits together and provides an extended fluid channel 30 structure between the inlet port 12 and the outlet port 14 in the filling assembly 10. Sealing joint 40 between the top of the filling assembly comprising the inlet port 12 and the bottom of the filling assembly comprising the outlet port 14 provides a fluid and air tight seal to prevent introduction or air or leakage of fluid during the pressure applied to direct fluid through the fluid channel structure and into the microtube connected to outlet port 14. A filter can optionally be placed between the two pieces for filtering sample fluid.

FIG. 5B is an isometric view of the bottom piece of the filling assembly shown in FIG. 5A, with a fluid channel 30 structure having a radial pattern of channels to direct sample fluid through the filling assembly.

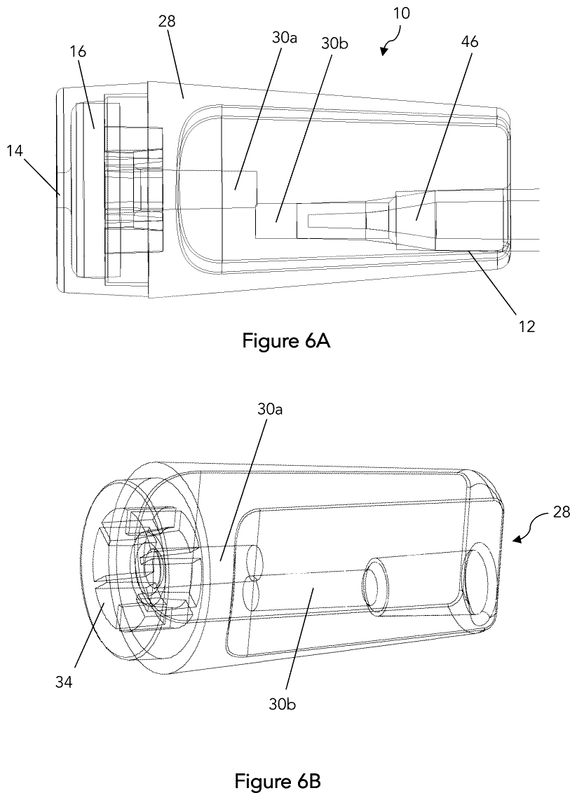

FIG. 6A is a side view of a two-piece filling assembly with an engaged sample transfer pipette 46 to transfer sample to the filling assembly 10 having a two-part fluid channel. The filling assembly 10 shown is comprised of two joined parts, a proximal part comprising the filter 16, and a distal part or filling assembly top 28 comprising the inlet port 12 to the fluid channel where the biological or sample fluid, such as blood, is applied. The proximal part comprising the filter 16 and filling assembly top 28 are joined with engagement features to provide a contiguous path from the fluid channel 30a, 30b to the filter 16. The bifurcated fluid channel 30a, 30b is shown here as a two-part fluid channel with the axis of the part of the fluid channel 30a proximal to the filter 16 offset relative to axis of the fluid channel 30b distal to the filter 16. The two bridging fluid channels 30a, 30b function in controlling the flow of fluid through the filling assembly 10 and down into the fluid conducting element, and optionally into the filter. The filtration and/or fluid transfer rate can affect the quality of the filtrate, so positioning an aperture or channel bifurcation or restriction feature between two fluid channels serves to modulate the fluid flow through the device. In particular, it is desirable to have controlled fluid application to the filling assembly and to prevent high pressure or spray-type fluid application as fluid pressure can introduce air into the fluid. In addition, high pressure fluid application to a filter can damage the filter and embed particulate in filter pores and clog the filter. An in-line aperture with an indirect fluid path to the filter prevents filter damage and limits any spray from passing through the filter.

FIG. 6B is a side perspective view of a filling assembly top 28 with engagement features 34 to engage with a filling assembly. Fluid channel segment 30a proximal to the filter 16 is shown offset relative to axis of the fluid channel 30b distal to the filter 16. The offset fluid channels 30a and 30b have a small overlap in the shape of a vesica piscis.

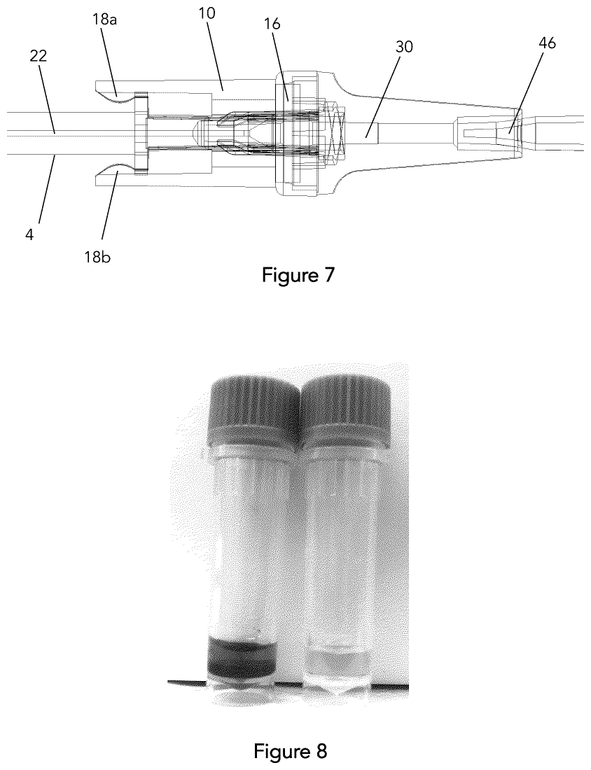

FIG. 7 is a side cross-sectional view of a filling assembly with engaged sample transfer pipette 46. Receiving syringe 4 is held in place relative to filling assembly 10 by syringe engagement features 18a, 18b. Filter 16 is secured in the filling assembly 10 and connected in line upstream to coaxial fluid channel 30 and downstream to a fluid conducting element 22 which directs fluid to the tip of receiving syringe 4.

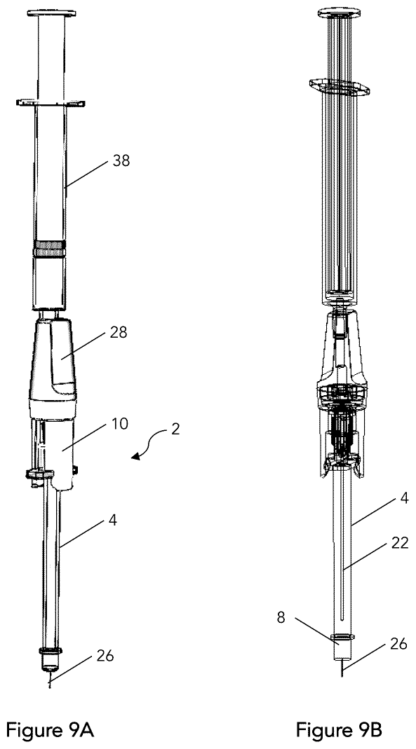

FIG. 9A is a side perspective view of a sample collection apparatus 2 with an engaged diluent syringe 38. Sample collection apparatus 2 has a filling assembly 10, filling assembly top 28 with fluid channel, and receiving syringe 4 with injection needle 26.

FIG. 9B is a side perspective transparent view of a sample collection apparatus with an engaged diluent syringe. As shown the diluent syringe has been expelled and pressure on the piston of the diluent syringe has forced diluent through the filter, through the fluid conducting element 22 and into the syringe tip 8 of the receiving syringe 4 of the sample collection apparatus. The diluted sample diluted with diluent from the diluent syringe can be injected onto an analytical device or automation device through the injection needle 26 for analysis. Appropriate dilution of biological or sample fluid can be tailored to the particular test being run on the diluted fluid sample.

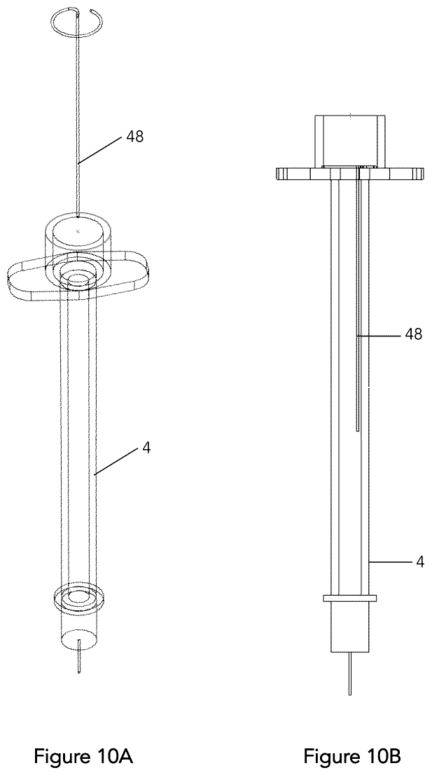

FIG. 10A is a side perspective exploded view of a receiving syringe 4 with pressure release structure 48, and FIG. 10B is a side perspective transparent view of a syringe with pressure release structure 48 inside the receiving syringe 4. Pressure release structure 48 provides an air escape mechanism during filling of the receiving syringe 8 through fluid conducting element 22. Other pressure release structure may be used including but not limited to one or more apertures, valves, seal obstructors, or combinations thereof.

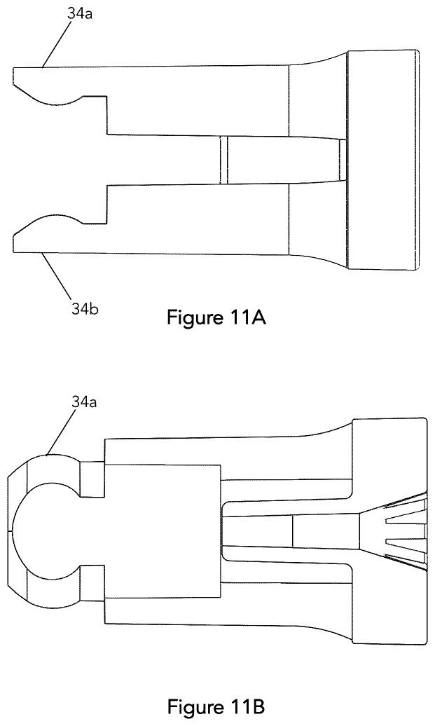

FIG. 11A is a side view of another embodiment of a sample collection apparatus and FIG. 11B is a side cross-sectional view of the sample collection apparatus in FIG. 11B. Engagement features 34a. 34b facilitate engagement of the filling assembly with a receiving syringe.

A kit can be provided comprising one or more a sample collection apparatus, pipette or other appropriate collection device, syringe plunger end or microplunger, and diluent syringe. The kit can optionally further be provided with, for example, a lancet for performing a fingerprick to obtain a blood sample, an alcohol wipe for cleaning off the finger prick site, and a bandage or cotton ball for closing the prick after the procedure. The kit could also further be provided with a control or test such that a full diagnostic kit can be provided as a complete diagnostic for one or more markers or the presence of one or more substances in the biological material.

The presently described sample collection apparatus and kit can be used in a method for separating and fluid handling of low volumes of components from low volumes of blood or other biological materials or sample fluids. In one example, the present apparatus and kit can be used for the separation of cells and large particles from plasma in whole blood. To obtain blood to be separated, the finger of a patient is cleaned, preferably using an alcohol swab, and the clean finger is then pricked with a provided lancet. Pressure is then applied to the pricked finger and blood is collected blood with a transfer pipette which is then placed inside the top of the sample collection apparatus housing. Preferably the transfer pipette is aligned with the fluid channel in the inlet port of the filling assembly housing. The sample is then dispensed vertically into the filling assembly of the sample collection apparatus and the diluent syringe is attached to the filling assembly. The plunger is then depressed to inject diluent onto the filter, and pressure is applied to the diluent syringe to push buffer-diluted plasma through filter and needle shaft into syringe tip. The diluent syringe is then removed from the filling assembly and the sample collection apparatus, and the syringe is separated from the microtube and filling assembly. A small microplunger can then be inserted into the syringe barrel such that the plunger can be used to inject small amounts of diluted plasma from the syringe into an analyzer. The syringe can also be used as is and loaded directly onto an analyzer for automated analysis of the diluted plasma sample.

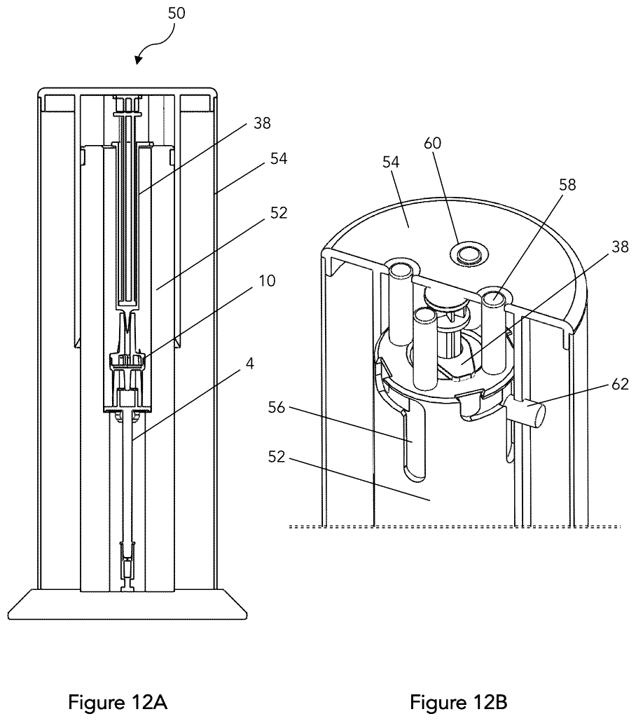

FIG. 12A is a cross-sectional view of a filling assist apparatus 50, which comprises an alignment column 52 and a weighted sleeve 54 which fits over and aligns with the alignment column 52. The fluid and optional filter inside the filling assembly 10 can be sensitive to pressure and standardizing the applied pressure of sample and diluent fluid onto the filter has been found to provide improved filtration results compared to manual pressure application onto the plunger of the diluent syringe 38. The filling assist apparatus 50 receives the sample collection apparatus with an engaged pressure application syringe into a hollow barrel in the alignment column 52 such that the flanges of the diluent syringe rest on the top of the alignment column and receiving syringe 4. Pressure application to the diluent syringe 38 by the weight of the weighted sleeve 54 provides the right amount of pressure to force the fluid through the filling assembly 10 without introducing air into the fluid or causing undue pressure on any of the components in the sample collection apparatus.

FIG. 12B is a close-up cross-sectional view of the top of a filter assist apparatus showing filling assist apparatus 50 for receiving a sample collection apparatus. Alignment column 52 comprises one or more guide track 56 for receiving an indicator pin 58 on the inside of the weighted sleeve 54. One or more pin aperture 60 on the weighted sleeve 54 provide a visual alignment mechanism such that a technician can properly align one or more guide pin 62 with pin aperture 60 to enable the weighted sleeve to apply the appropriate amount of pressure to the sample collection apparatus inside the filling assist apparatus 50.

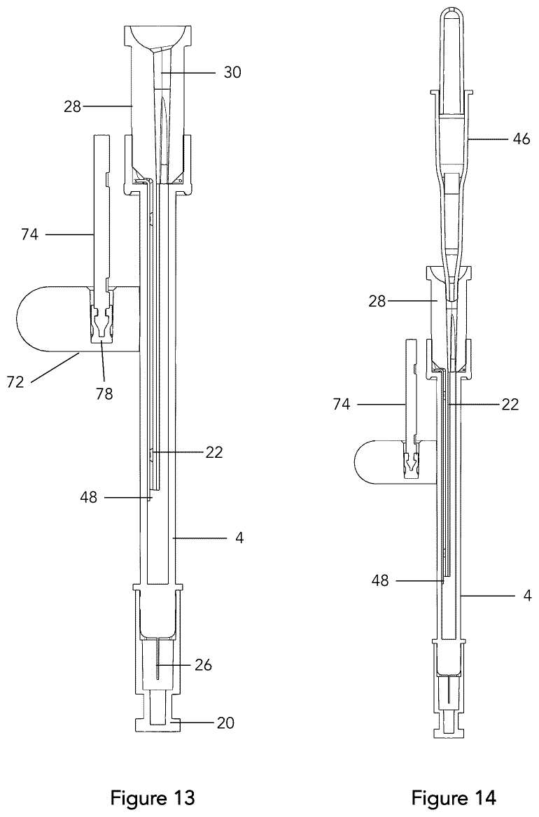

FIG. 13 is a side cross sectional view of an alternative embodiment of a fluid sample collection apparatus. A filling assembly 28 having a filling assembly fluid channel 30 is engaged with a receiving syringe 4. The engagement of the filling assembly 28 to the receiving syringe can be by one of various engagement mechanisms, including but not limited to friction-fitting, pressure-fitting, or combination thereof, optionally with a releasable locking mechanism. The filling assembly 28 may be of varying size to accommodate differing volumes of biological sample and receiving syringe diameters. Similarly, fluid channel 30 may be of varying diameters to fit with application devices such as transfer pipettes or transfer syringes of varying diameters. In an example, a cone-shaped filling assembly 28 having a filling assembly fluid channel 30 that is widest at the region distal to the engaged receiving syringe 4 may limit the possibility of sample spillage or overflow at the application stage into fluid channel 30 by increasing the channel volume at the sample receiving opening for applying sample.

A fluid conducting element 22 in receiving syringe 4 is fluidly connected to the fluid channel 30 in the filling assembly 28 and configured to accommodate the passage of sample fluid through the receiving syringe 4 and toward the injection needle 26. In the present embodiment, the fluid conducting element 22 is a channel guide that sits snugly inside the channel of receiving syringe 4 such that surfaces of the channel guide or fluid conducting element 22 make continuous contact with the inside wall of the receiving syringe 4. The inside wall of the receiving syringe 4 together with the fluid conducting element 22 creates a substantially fluid-tight fluid flow channel where sample can flow from the fluid channel 30 down the receiving syringe 4. This embodiment of the fluid flow channel is formed primarily from curvatures along the fluid conducting element 22, which in this embodiment is a connected and contiguous extension of the fluid channel 30 of the filling assembly 28. However, other fluid conducting elements may be adapted to this region of the device and are considered to be within the scope of the present invention. For example, tubes having a complete channel as well as elongated devices that form a channel together with the sides of the receiving syringe can serve as fluid conducting elements and can be of varying lengths and diameters to accommodate fluid flow into the receiving syringe 4. The fluid conducting element can also be connected or reversibly connectable to the fluid assembly 28. The fluid conducting element acts by facilitating the passage of biological fluid from the filling assembly fluid channel 30 through the receiving syringe 4 and toward the injection needle 26. In another example, the fluid conducting element 22 may consist of a simple barrier that traverses a substantive length of the receiving syringe 4 and separates it into two or more channels. A needle cap 20 covers the injection needle 26 to enhance safety when the sample collection apparatus is not in use. The needle cap 20 may be screwed into position or secured over the injection needle 26 by a friction fit with along a circumference of the receiving syringe 4. Needle cap protects injection needle 26 during loading of sample into the filling assembly 28 and receiving syringe 4 and is removed prior dispensing of sample from the receiving syringe. A pressure release structure 48 extends along the inside of receiving syringe 4 to facilitate the escape of air that would otherwise accumulate as biological fluid is guided along the fluid conducting element within the receiving syringe.

The sample collection apparatus can also be supplied with a microplunger 74 which is used in compressing biological fluid within the receiving syringe 4 when the fluid assembly 28 and fluid conducting element 22 is disengaged from the receiving syringed. The microplunger 74 can be provided in a kit with the sample collection apparatus, or as shown as releasably secured to the sample collection apparatus by means of an attachment clip 72. The microplunger 74 may be comprised of any solid material that is strong enough to withstand the pressures associated with pushing compressed air and viscous biological fluids, such as blood, through the receiving syringe 4 and injection needle 26. The length of the microplunger 74 should be at least long enough that it can be engaged by a rod or other device inserted into the inside barrel of the receiving syringe, but may be shorter than the length of the syringe barrel as shown, or longer than the syringe barrel as desired. The diameter of the plunger tip 78 of the microplunger 74 should be wide enough that a fluid seal can be created between the plunger tip 78 and the inside of receiving syringe 4 and sufficient that the plunger tip 78 can be slid down the syringe body to eject sample through injection needle 26.

FIG. 14 is a side cross-sectional view of a sample collection apparatus with a sample transfer pipette 46 reversibly engaged with a filling assembly 28, which, in turn, is engaged with the receiving syringe 4. Biological fluid originating within the sample transfer pipette 46 is dispensed into the fluid channel 30 of the filling assembly 28 by pressure applied to the sample transfer pipette 46. The end of sample transfer pipette 46 is received in the fluid channel 30 with the end of sample transfer pipette 46 aligned close to the such that sample can be deposited close to start of fluid conducting element 22, which in this embodiment is a channel guide. Once all of the sample has been ejected into the fluid channel, excess air in the sample transfer pipette 46 applies pressure to the fluid sample pushing down fluid conducting element 22 and towards the bottom of receiving syringe 4. Additional air pressure via the sample transfer pipette 46 or other device can be additionally applied to the fluid channel 30 to further move the fluid sample down the fluid conducting element 22 and into the bottom of the syringe. As fluid and air pressure are applied from the sample transfer pipette 46 air can escape the receiving syringe 4 through an air channel created by abutment of the channel guide (fluid conducting element 22) with the inside walls of the receiving syringe and kept open by pressure release structure 48. Once sample fluid has been received in the bottom of receiving syringe 4, the fluid conducting element 22 and filling assembly 28 can be removed from the receiving syringe 4 such that it can be used for ejecting the sample through the injection needed. The microplunger 74 can then be inserted into the inside of receiving syringe and pressure applied will, once the plunger tip is pushed below the pressure release structure 48, will apply pressure to eject the sample fluid through the injection needle. The sample transfer pipette 46 may be a traditional transfer pipette as depicted, however any other sterile means of transferring sample fluid into the fluid channel 30 of the filling assembly 28 is contemplated to be within the scope of the present invention. For example, serological pipettes and micropipettes may also be used in the sample application stage.

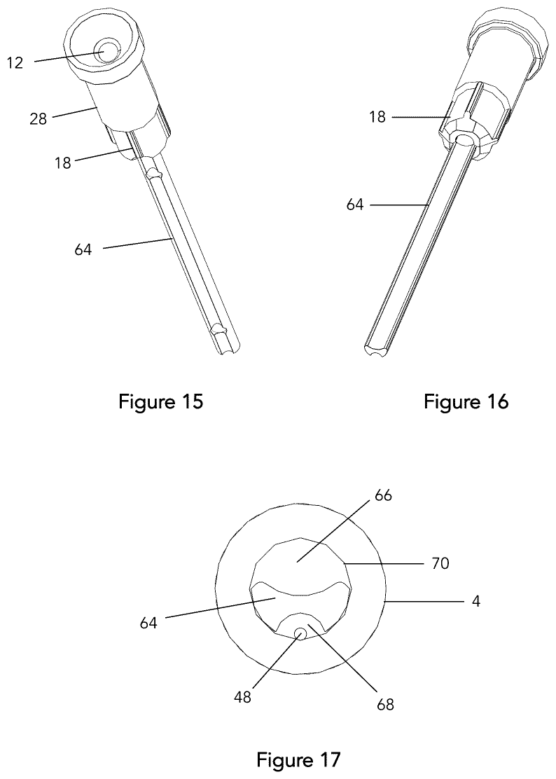

FIG. 15 is a top isometric view of a one-piece fluid conducting element and filling assembly. Channel guide 64 acts as a fluid conducting element when inserted into a receiving syringe, together with the filling assembly 28 with a fluid channel. The channel inlet port 12 is an opening to the fluid channel within the filling assembly 28 configured to receive the dispensing end of a sample transfer pipette or other sterile means of biological fluid transfer. The channel inlet port 12 may be variable in diameter to accommodate different modes or rates of fluid transfer, and differently sized transfer pipettes. Preferably, the sample transfer pipette engages with the fluid channel and directs sample fluid directly adjacent the bottom of the filling assembly 28 where the outlet port of the filling assembly 28 meets the fluid conducting element. The shape, size, and length of the fluid channel and inlet port 12 can be sized to receive a particular sample transfer pipette to prevent lateral movement of the sample transfer pipette and direct sample fluid in the most desirable location adjacent the channel guide 64 or fluid conducting element. Various channel guide 64 lengths are contemplated with, for example, longer channel guides 64 being better suited to longer receiving syringes 4. In some embodiments, a channel guide 64 may be reversibly extendable, to accommodate various lengths of receiving syringe 4. Channel guides 64 may be made from any solid, sterile (or sterilizable) material that is conducive to fluid passing along it such that it would not compromise or contaminate the biological sample under scrutiny. Engagement features 18, shown here as friction fit protrusions, assist in securing the filling assembly 28 to the receiving syringe. Other engagement features can also be used, including but not limited to rubberized septa, screw-type engagement features, and other configurations of friction-fit engagement features.

FIG. 16 is a bottom isometric view of a channel guide 64 which serves as a fluid conducting element and filling assembly 28. A syringe engagement feature 18 is present in the form of protrusions along an end of the filling assembly 28 proximal to the start of channel guide 64. In this embodiment, the syringe engagement feature 18 relies on a friction-fit to secure the filling assembly 28 to the receiving syringe. In the depicted embodiment, this friction fit consists of narrow protrusions that emanate radially from the filling assembly 28 and engage with the receiving syringe 4, although other mechanisms of engagement are contemplated to be within the scope of the present invention.

FIG. 17 is a cross-sectional view of channel guide 64 which serves as a fluid conducting element 22 in the receiving syringe 4. The channel guide 64 is configured to create a fluid flow channel 66 and an air flow channel 68 when engaged with the inner wall 70 of the receiving syringe. When the channel guide 64 is inserted into the receiving syringe 4, a fluid flow channel 66 and an air flow channel 68 are formed on either side of the channel guide 64. This allows air to escape from the receiving syringe, which can be of very low volume, such as less than 1 mL, while sample fluid is inserted through the fluid flow channel 66. Providing a channel for air escape also prevents the introduction of air bubbles during sample loading of the syringe. The channel guide 64 shown is contoured to engage on two sides with the inner wall 70 of the receiving syringe 4 to provide a substantially fluid tight seal against the inner wall 70. When the filling assembly 28 and channel guide 64 are engaged with the receiving syringe, sample can be directed through the filling assembly fluid flow channel 66 while air is allowed to escape through the air flow channel 68 preventing air pressure buildup inside the receiving syringe. Pressure release structure 48 inside the air flow channel 68 maintains an open airway between the engaged filling assembly and the open end of the receiving syringe to allow air to escape during sample fluid application down the fluid flow channel 66. The fluid flow channel 66 and air flow channel 68 may adopt various shapes and sizes depending on the diameter of the receiving syringe 4 and the configuration of the channel guide 64. For example, the contours of the channel guide 64 may be symmetrical to produce a fluid flow channel 66 and an air flow channel 68 that are the same size. Such a configuration could improve case of use by eliminating any possibility of incorrectly engaging the filling assembly 28 and attached channel guide 64 with the receiving syringe 4. Alternatively, the contours of the channel guide 64 may be asymmetrical to maximize the rate of fluid flow into the receiving syringe and toward the injection needle, while minimizing the volume in the air flow channel 68 used to separate the pressure release structure 48 from contact with biological fluid.

The pressure release structure 48 separates the filling assembly 28 from the receiving syringe at a location along the point of engagement to facilitate the escape of air that would otherwise pressurize within the receiving syringe as the microplunger is depressed. In the depicted embodiment, the pressure release structure 48 is comprised of a thin wire that encircles the circumference of the top of the receiving syringe 4 and extends along the inside of the receiving syringe and terminates at a location above the injection needle. The pressure release structure 48 should terminate at a high sufficiently above the injection needle such that a microplunger can clear the end of the injection needle without substantially interacting with the sample fluid that has collected at the bottom of the receiving syringe. In one example, the pressure release structure 48 terminate between 1 mm and 10 mm above the sample fluid level at the bottom of the syringe, or above the level of the maximum sample volume that the syringe is designed to hold. Other pressure release structures that can be used to generate a small gap between the engaged filling assembly 28 and receiving syringe 4 are also contemplated to be within the scope of the present invention.

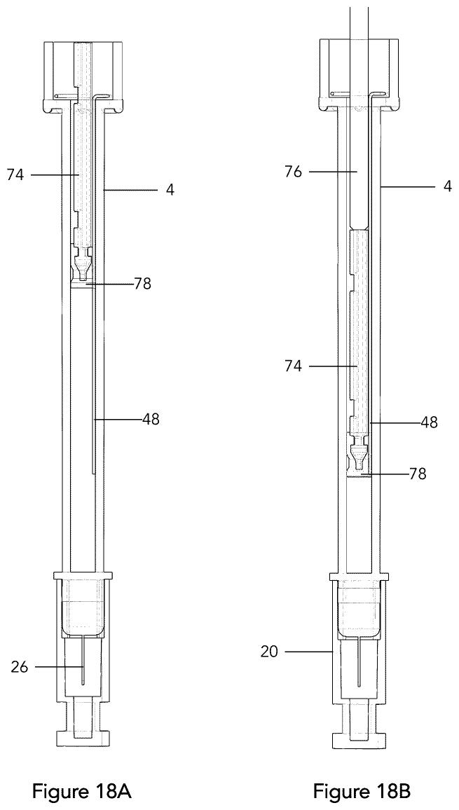

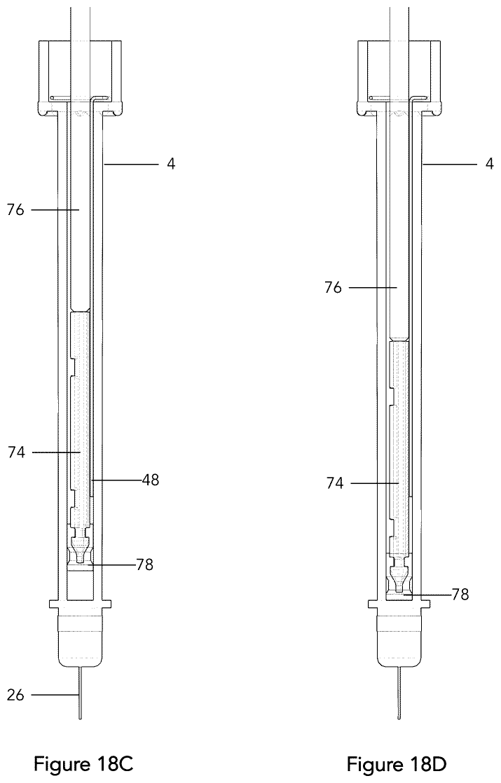

FIGS. 18A-18D show a method for using the present syringe filling apparatus for loading a receiving syringe with an injection needle.

FIG. 18A is a side cross-sectional view of a receiving syringe 4 with an engaged microplunger 74. After sample has been injected into the filling assembly and directed down the fluid conducting element, the filling assembly and directed down the fluid conducting element can be removed from the receiving syringe 4. At this stage the sample fluid will have been collected in the bottom of the sample receiving syringe 4 adjacent the injection needle 26. The microplunger 74 engages with the inner wall of the receiving syringe 70 at a plunger tip 78, forming a substantially fluid-tight connection with the inner wall of the receiving syringe, except at the location around the plunger tip 78 seal where pressure release structure 48 is present. The plunger tip 78 may be comprised of any material that can be compressed to generate an airtight seal while retaining mobility along the inner walls of the receiving syringe 70. As the microplunger 74 is depressed and moved along the inner wall of the receiving syringe 4 towards the injection needle, the plunger tip 78 presses against the pressure release structure 48, which extends along the inner wall of the receiving syringe 4. Any air that pressurizes within the receiving syringe 4 as the microplunger 74 is depressed can escape via the pressure release structure 48 as long as the plunger tip 78 of the microplunger 74 is in contact with the pressure release structure 48.

FIG. 18B is a side cross-sectional view of a receiving syringe 4 with engaged microplunger 74 and injection rod 76. The injection rod 76, which is preferably a component of an automated injection system and part of an automated sample analyzer, allows the plunger tip 78 of the microplunger 74 to be depressed to the end of the pressure release structure 48 and thereby prepares the sample collection apparatus for dispensing biological fluid that has been previously applied to the receiving syringe 4. Ideally, the level of biological fluid to be dispensed is below the end of pressure release structure 48 such that sample fluid is not significantly displaced while the microplunger is moved toward the injection needle in preparing for dispensing the sample through the dispensing needle. An air pocket of pressurized air can be retained between the plunger tip 78 of the microplunger 74 and the sample when dispensing. The injection rod can be moved, for example, using a stepper motor or other motor by the automated injection device in the analyzer. A needle cap 20 prevents any leakage of biological fluid during the preparatory phases of sample application and insertion of the microplunger 74 via the injection rod 76.

FIG. 18C is a side cross-sectional view of a receiving syringe 4 with engaged microplunger 74 and injection rod 76 with the plunger tip 78 below the end of the pressure release structure 48. The injection rod 76 is extended further into the receiving syringe such that the plunger tip 78 of the microplunger 74 is adjacent to or near the previously applied biological fluid. Pressure on the microplunger 74 via injection rod 76 results in sample being ejected through the injection needle 26. At this position, the plunger tip 78 is allowed to make an airtight seal at all points along the inner wall of the receiving syringe 70 since air leakage is no longer assisted by pressure release structure 48. In an automated injection device or automated analyzer device, injection rod can be moved down using, for example, a stepper motor capable of fine motion to eject ultra-small sample volumes onto a sample receiving device such as an analyser membrane. In practice, the present device can dispense volumes in the sub-microlitre range, such as between, for example, 0.1 μL and up. In one example, the amount of sample dispensed by the injection needle 26 is between about 0.1 μL and 2.5 μL. Depression of the microplunger 74 toward the injection needle 26 ejects an amount of biological fluid that is commensurate with the distance moved by the microplunger 74.

FIG. 18D is a side cross-sectional view of a receiving syringe 4 with engaged microplunger 74 and injection rod 76 in a fully deployed state. In this position, the plunger tip 78 of the microplunger 74 extends as far into the receiving syringe 4 as possible and the dispensing of biological fluid is complete. The injection rod 76 can then be manually removed from the receiving syringe 4, in one example by removing the receiving syringe 4 from the automated device which controls the injection rod.

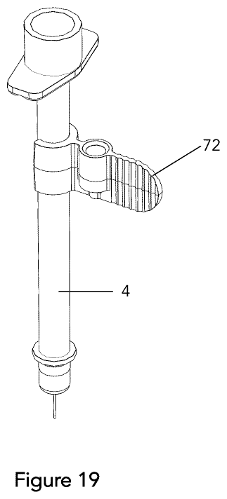

FIG. 19 is a top isometric view of a receiving syringe 4 with attachment clip for holding a microplunger. The attachment clip 72 may be reversibly or permanently connected to the receiving syringe and can be comprised of any solid material that is capable of holding the microplunger in place for easy access by the user of the device. Various approaches can be used to connect the attachment clip 72 to the body of the receiving syringe 4. For example, the attachment clip 72 may be spring-loaded with jaws that are biased in a closed position and pinch around the receiving syringe. Alternatively, the attachment clip 72 may have an end that is conveniently curved to fit tightly around the circumference of the receiving syringe 4. In other embodiments, the attachment clip may have multiple recesses for holding one or more additional tools.

The presently described syringe filter was used to collect diluted plasma from a whole blood sample. Two aliquots of blood were collected from three separate donors. The first aliquot was processed using the sample collection apparatus as follows. To collect the sample using the blood collection device, whole blood was collected from the donor via a finger prick to the ring-finger of the non-dominant hand. The tip of a blood collection device or pipette used to collect the blood and the collection device was placed into the entry port of the sample collection apparatus. The whole blood sample was then dispensed into the sample collection apparatus and the whole blood was allowed to incubate within the sample collection apparatus for a couple of minutes. A diluent syringe was then engaged with the entry port of the sample collection apparatus. The diluent syringe contains the buffer used to elute the plasma/serum from the sample collection apparatus, while it leaves the hematocrit within the sample collection apparatus. The plunger on the diluent syringe was depressed until the entirety of the buffer was dispensed into the sample collection apparatus. The resulting filtrate collected from the other side of the filter is clear and slightly pinkish in colour and devoid of any visible hematocrit. The resulting filtrate and the second collected aliquot of whole blood were then centrifuged at 1300×g for 10 mins to pellet the hematocrit. The centrifuged samples were then compared to evaluate the level of hematocrit depletion achieved by the sample preparation protocol using the sample collection apparatus. The results are shown in Table 1. In all three cases, substantial hematocrit remains in the samples that did not undergo sample preparation protocol using the sample collection apparatus. FIG. 8 is an image of a whole blood and filtered blood sample prepared using the described method.

TABLE 1

| | |
|---|---|
| Filtered | Whole blood non-filtered |

| Sample | Total | Height | Total | Height |
|---|---|---|---|---|
| No. | height | of RBCs | height | of RBCs |
| 1 | 8 mm | 0 mm | 8 mm | 4 mm |
| 2 | 5 mm | 0 mm | 5 mm | 3 mm |
| 3 | 6 mm | 0 mm | 6 mm | 3 mm |

A total hematocrit in each sample was determined by measuring the volume percentage (vol %) of red blood cells (RBC) in blood. The total height is the total height of the column of filtered sample or centrifuged sample; both samples had the same volume and therefore have the same height within the sample tube. The height of the red blood cells (RBCs) is the height of the column of the hematocrit within the total height of the column. For example, if the total height of the column is 8 mm and the RBC height is 4 mm, then there is approximately 50% hematocrit within the sample. If the total height of the column is 8 mm and the RBC height is 0 mm, then there is no visible hematocrit within the sample. As shown in Table 1, whole blood filtered with the presently described device is substantially free of blood cellular material.

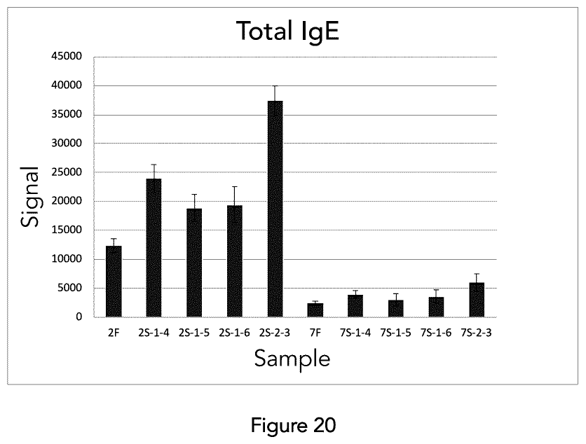

A comparison of total IgE levels in plasma samples was compared between plasma collected from blood using the presently described device and protocol and plasma samples collected by centrifugation. Two blood Donors were sampled, Donors 2 and 7. Blood collected using a finger prick collection (between 20-200 μl is typical), samples (2F, 7F), was processed using the presently described device by using a pipette to collect the blood and placing the collected blood into the entry port and fluid channel of the sample collection apparatus. The whole blood was allowed to incubate inside the fluid channel of the sample collection apparatus for two minutes. A diluent 1X phosphate buffered saline (PBS) buffer syringe was then engaged with the entry port of the sample collection apparatus during which the operator and diluent was ejected into the sample collection apparatus to elute the plasma/serum from the sample collection apparatus, while leaving the majority of the hematocrit within the sample collection apparatus. The entirety of the buffer in the diluent syringe was depressed until the contents of the diluent syringe was dispensed into the sample collection apparatus, which resulted in a predetermined volume of filtrate to be dispensed into the receiving syringe. The resulting filtrate was clear and slightly pinkish in colour. Blood was also collected in a venous blood draw, samples (2S, 7S). Venous-collected blood was centrifuged at 2.5 Xg for 10 minutes to provide clear plasma, which was then diluted with 1×PBS to produce a titration curve (1:4, 1:5, 1:6, 2:3). Diluted plasma samples were applied to the assay platform, read on a laboratory reader and then analyzed using the research software. Total IgE levels for Donor 2 (199 kU/L) and Donor 7 (32 kU/L) were identified by a reference laboratory. FIG. 20 is a graph displaying the measured IgE results displayed as fluorescent units as provided by the lab reader and the ImmunoCap (Thermofisher Phadia) platforms. The results demonstrate that total IgE can be measured accurately using the presently described device and method where the blood is collected using a finger prick and using a venous whole blood sample with a well-established laboratory IgE quantification system (ImmunoCap).

All publications, patents and patent applications mentioned in this specification are indicative of the level of skill of those skilled in the art to which this invention pertains and are herein incorporated by reference. The invention being thus described, it will be obvious that the same may be varied in many ways. Such variations are not to be regarded as a departure from the scope of the invention, and all such modifications as would be obvious to one skilled in the art are intended to be included within the scope of the following claims.

1. A sample collection apparatus comprising:
a filling assembly comprising:
an inlet port for receiving a sample fluid; and
an outlet port fluidly connected to the inlet port by a fluid channel;
a receiving syringe having a first end with an aperture for engaging with the filling assembly and a second end comprising an injection needle;
a fluid conducting element inside the receiving syringe extending from the outlet port of the filling assembly toward the second end of the receiving syringe adjacent the injection needle to direct fluid to the bottom of the receiving syringe; and
a pressure release structure in the receiving syringe to release pressure in the receiving syringe as sample fluid flows via the fluid conducting element.

2. The apparatus of claim 1, wherein the inlet port in the filling assembly is sized to fluidly connect with a transfer pipette for receiving sample fluid under pressure.

3. The apparatus of claim 1, further comprising a filter in the filling assembly disposed between the inlet port and the outlet port.

4. The apparatus of claim 1, wherein the receiving syringe volume is 1 mL or less.

5. The apparatus of claim 1, wherein the receiving syringe diameter is 4 mm or less.

6. The apparatus of claim 1, wherein the inlet port in the filling assembly is tapered.

7. The apparatus of claim 1, wherein the fluid channel in the filling assembly comprises a flow restrictor.

8. The apparatus of claim 1, wherein the fluid conducting element comprises a hollow shaft.

9. The apparatus of claim 1, wherein the fluid conducting element comprises a channel guide which, together with a sidewall of the receiving syringe, forms a fluid channel.

10. The apparatus of claim 1, wherein the fluid conducting element comprises a polymer, metal, or a combination thereof.

11. The apparatus of claim 1, wherein the fluid conducting element creates a fluid flow channel with a cross-sectional diameter of between about 0.05 and 3.0 mm.

12. The apparatus of claim 1, wherein the filling assembly engages with the receiving syringe via a friction fit feature, snap-fit feature, a flange-locking feature, or a combination thereof.

13. A sample collection kit comprising:
a filling assembly comprising:
an inlet port for receiving a sample fluid; and
an outlet port fluidly connected the inlet port by a fluid channel;
a receiving syringe having a first end with an aperture for engaging with the filling assembly and a second end comprising an injection needle, and an inside wall;
a fluid conducting inside the receiving syringe extending from the outlet port of the filling assembly toward the second end of the receiving syringe adjacent the injection needle to direct fluid to the bottom of the syringe;
a pressure release structure in the receiving syringe to release pressure in the receiving syringe as sample fluid flows via the fluid conducting element; and
a microplunger having a plunger tip for creating a fluid-tight seal with the inside wall of the receiving syringe.

14. The kit of claim 13, further comprising a diluent syringe.

15. The kit of claim 13, further comprising a fluid sample transfer pipette having a tip configured to form a fluid tight connection with the inlet port of the filling assembly.

16. The kit of claim 13, further comprising a lancet.

17-18. (canceled)

19. The apparatus of claim 1, wherein the pressure release structure comprises a thin wire that extends along the inside of the receiving syringe and terminates at a location above the injection needle.

20. The apparatus of claim 1, wherein the pressure release structure allows air to escape during filling of the receiving syringe.

21. The apparatus of claim 1, wherein the fluid conducting element makes continuous contact with the inside wall of the receiving syringe.

22. The apparatus of claim 1, wherein the fluid conducting element creates a substantially fluid-tight fluid channel where sample can flow from the first end of the receiving syringe through the fluid channel to the second end of the receiving syringe.
